(19) United States
(12) Patent Application Publication
(10) Pub. No.: US 2024/0423933 A1
(43) Pub. Date: Dec. 26, 2024

(54) DRUGS FOR TREATING NEURODEVELOPMENTAL DISORDERS

(71) Applicants: President and Fellows of Harvard College, Cambridge, MA (US); Trustees of Tufts College, Medford, MA (US)

(72) Inventors: Richard Novak, Cambridge, MA (US); Frederic Vigneault, Cambridge, MA (US); Michael Levin, Beverly, MA (US); Donald E. Ingber, Cambridge, MA (US)

(73) Assignees: President and Fellows of Harvard College, Cambridge, MA (US); Trustees of Tufts College, Medford, MA (US)

(21) Appl. No.: 18/561,989

(22) PCT Filed: May 20, 2022
(86) PCT No.: PCT/US2022/030372

§ 371 Date: Nov. 17, 2023

Related U.S. Application Data
(60) Provisional application No. 63/191,821, filed on May 21, 2021.

(51) Int. Cl. A61K 31/167; A61K 31/365; A61K 31/40; A61K 35/00; A61K 35/74; A61P 25/00; A61P 25/22

(52) U.S. Cl. CPC A61K 31/167; A61K 31/365; A61K 31/40; A61K 35/74; A61P 25/00; A61P 25/22; A61K 2035/11

(57) ABSTRACT

The present disclosure provides compositions and methods for treating neurodevelopmental disorders, such as Rett syndrome and cyclin-dependent kinase-like 5 (CDKL5) deficiency disorder.

### RELATED APPLICATION

This application is a national stage filing under 35 U.S.C. § 371 of international patent application number PCT/US2022/030372, filed May 20, 2022, which claims the benefit under 35 U.S.C. § 119 (c) of U.S. provisional application Ser. No. 63/191,821, filed May 21, 2021, the content of each of which is incorporated by reference herein in its entirety.

### BACKGROUND

Rett syndrome (RTT), a neurodevelopmental disorder that primarily affects females is typically characterized by loss of language skills and hand use, impaired or absent gait, dyspraxia, cognitive deficits, stereotyped behaviors, seizures, and autonomic irregularities including respiratory and gastrointestinal (GI) dysfunction and premature osteoporosis and osteopenia (Hagberg et al. Ment. Retard. Dev. Disabil. Res. Rev. 2002; 8:61-65; Operto et al. Brain Behav. 2019; 9: e01250). Mutations in the X-linked gene encoding MECP2 (methyl-CpG-binding protein 2) account for 90-95% of the case of classic Rett syndrome (RTT) (Neul et al. Neuroscientist. 2004; 10:118-128; Neul et al. Neurology. 2008; 70:1313-1321) while mutations in the X-linked gene encoding cyclin-dependent kinase-like 5 (CDKL5) account from some cases of atypical RTT that manifest with early refractory epilepsy (Olson et al. Pediatr. Neurol. 2019; 97:18-25). CDKL5 deficiency disorder (CDD) has overlapping phenotypic features with RTT including seizures and developmental delays, GI dysfunction, scoliosis, limited or absent speech, and sleep disturbances. However, individuals with CDD exhibit severe developmental delay from birth and seizure onset before the age of 3 months (Fehr et al. Neurology. 2016; 87:2206-2213; Fehr et al. Eur. J. Hum. Genet. 2013; 21:266-273). Seizures and sleep disturbances are more common in CDD than in RTT, whereas features of regression and spinal curvature are less common in those with CDKL5 mutations compared to those with MECP2 mutations (Fehr et al. Eur. J. Hum. Genet. 2013; 21:266-273; Tarquinio et al. Brain. 2017; 140:306-318).

While some treatments for RTT and CDD are in clinical trials, nearly all were not designed explicitly for the genetic disorder but rather are being repositioned based on clinical endpoint similarity to previously-tested indications and have demonstrated only mild efficacy. Additionally, since the predominant endpoint being pursued has been seizures and some neuromuscular deficiencies, other aspects of RTT and CDD, including gastrointestinal tract and inflammatory disruptions, are still in need of a concerted therapeutic effort.

### SUMMARY

Some aspects of the present disclosure provide a method of treating a symptom of Rett syndrome in a subject in need thereof, the method comprising administering to the subject a therapeutically effective amount of vorinostat.

In some embodiments, the subject administered vorinostat has been diagnosed with Rett syndrome.

In some embodiments, the vorinostat is formulated with hydroxypropyl B-cyclodextrin. In some embodiments, the amount of vorinostat is effective to reduce anxiety in the subject, relative to a control. In some embodiments, the amount of vorinostat is effective to improve Rett syndrome scoring of the subject, relative to a control. In some embodiments, the amount of vorinostat is effective to decrease inflammation (e.g., inflammation in the gastrointestinal tract) in the subject, relative to a control. In some embodiments, the amount of vorinostat is effective reduce, inhibit, or reverse ciliary dysfunction in the subject, relative to a control.

In some embodiments, the amount of vorinostat is lower than the amount of vorinostat approved by the U.S. Food and Drug Administration (FDA) for the treatment of cutaneous T cell lymphoma (CTCL).

In some embodiments, the vorinostat is administered to the subject in combination with one or more of ivermectin, trofinetide, and Bacteroides fragilis or a polysaccharide isolated from Bacteroides fragilis.

Yet other aspects of the present disclosure provide a method of treating a symptom of Rett syndrome in a subject in need thereof, the method comprising administering to the subject a therapeutically effective amount of ivermectin.

In some embodiments, the subject administered ivermectin has been diagnosed with Rett syndrome.

In some embodiments, the amount of ivermectin is effective to reduce or inhibit seizures in the subject.

In some embodiments, the ivermectin is administered to the subject in combination with one or more of vorinostat, trofinetide, and Bacteroides fragilis or a polysaccharide isolated from Bacteroides fragilis.

Still other aspects of the present disclosure provide a method of treating a symptom of Rett syndrome in a subject in need thereof, the method comprising administering to the subject a therapeutically effective amount of Bacteroides fragilis or a polysaccharide isolated from Bacteroides fragilis.

In some embodiments, the subject administered Bacteroides fragilis or a polysaccharide isolated from Bacteroides fragilis has been diagnosed with Rett syndrome. In some embodiments, the Bacteroides fragilis is Bacteroides fragilis 9343. In some embodiments, the method comprises administering to the subject a therapeutically effective amount of Bacteroides fragilis. In some embodiments, the method comprises administering to the subject a therapeutically effective amount of a polysaccharide isolated from Bacteroides fragilis. In some embodiments, the polysaccharide is polysaccharide A (PSA). In some embodiments, the polysaccharide is polysaccharide B (PSB).

In some embodiments, the Bacteroides fragilis or a polysaccharide isolated from Bacteroides fragilis is administered to the subject in combination with one or more of vorinostat, ivermectin, and trofinetide.

Some aspects of the present disclosure provide a method of treating a symptom of CDKL5 deficiency disorder in a subject in need thereof, the method comprising administering to the subject a therapeutically effective amount of vorinostat.

In some embodiments, the subject administered vorinostat has been diagnosed with CDKL5 deficiency disorder.

In some embodiments, the vorinostat is formulated with hydroxypropyl B-cyclodextrin.

In some embodiments, the amount of vorinostat is effective to reduce anxiety in the subject, relative to a control. In some embodiments, the amount of vorinostat is effective to decrease inflammation (e.g., inflammation in the gastrointestinal tract) in the subject, relative to a control. In some embodiments, the amount of vorinostat is effective reduce, inhibit, or reverse ciliary dysfunction in the subject, relative to a control.

In some embodiments, the vorinostat is administered in combination with one or more of ivermectin, trofinetide, and Bacteroides fragilis or a polysaccharide isolated from Bacteroides fragilis.

Other aspects of the present disclosure provide a method of treating a symptom of CDKL5 deficiency disorder in a subject in need thereof, the method comprising administering to the subject a therapeutically effective amount of ivermectin.

In some embodiments, the subject administered ivermectin has been diagnosed with CDKL5 deficiency disorder.

In some embodiments, the amount of ivermectin is effective to reduce or inhibit seizures in the subject.

In some embodiments, the ivermectin is administered in combination with one or more of vorinostat, trofinetide, and Bacteroides fragilis or a polysaccharide isolated from Bacteroides fragilis.

Still other aspects of the present disclosure provide a method of treating a symptom of CDKL5 deficiency disorder in a subject in need thereof, the method comprising administering to the subject a therapeutically effective amount of trofinetide.

In some embodiments, the subject administered trofinetide has been diagnosed with CDKL5 deficiency disorder.

In some embodiments, the amount of trofinetide is effective to reduce or inhibit seizures in the subject.

In some embodiments, the trofinetide is administered in combination with one or more of vorinostat, ivermectin, and Bacteroides fragilis or a polysaccharide isolated from Bacteroides fragilis.

Further aspects of the present disclosure provide a method of treating a symptom of CDKL5 deficiency disorder in a subject in need thereof, the method comprising administering to the subject a therapeutically effective amount of Bacteroides fragilis or a polysaccharide isolated from Bacteroides fragilis.

In some embodiments, the subject administered Bacteroides fragilis or a polysaccharide isolated from Bacteroides fragilis has been diagnosed with CDKL5 deficiency disorder. In some embodiments, the Bacteroides fragilis is Bacteroides fragilis 9343. In some embodiments, the method comprises administering to the subject a therapeutically effective amount of Bacteroides fragilis. In some embodiments, the method comprises administering to the subject a therapeutically effective amount of a polysaccharide isolated from Bacteroides fragilis. In some embodiments, the polysaccharide is PSA. In some embodiments, the polysaccharide is PSB. In some embodiments, the polysaccharide is as described in Zheng, L. et. al., Capsular Polysaccharide From Bacteroides fragilis Protects Against Ulcerative Colitis in an Undegraded Form. Front. Pharmacol., 7 Dec. 2020 doi.org/10.3389/fphar.2020.570476.

In some embodiments, the Bacteroides fragilis or a polysaccharide isolated from Bacteroides fragilis is administered in combination with one or more of vorinostat, ivermectin, and trofinetide.

In some embodiments, the vorinostat is administered weekly, daily, or multiple times a day. In some embodiments, the ivermectin is administered weekly, daily, or multiple times a day. In some embodiments, the trofinetide is administered weekly, daily, or multiple times a day. In some embodiments, the Bacteroides fragilis or a polysaccharide isolated from Bacteroides fragilis is administered weekly, daily, or multiple times a day.

Other aspects of the present disclosure provide a method of treating a symptom of a neurodevelopmental disorder in a subject in need thereof, the method comprising administering to the subject a therapeutically effective amount of an agent of Table 1.

In some embodiments, the neurodevelopmental disorder is Rett syndrome. In other embodiments, the neurodevelopmental disorder is CDKL5 deficiency disorder.

Also provided herein are pharmaceutical compositions comprising (a) two or more of vorinostat, ivermectin, trofinetide, and Bacteroides fragilis or a polysaccharide isolated from Bacteroides fragilis and (b) a pharmaceutically acceptable excipient. In some embodiments, the excipient is hydroxypropyl B-cyclodextrin.

### BRIEF DESCRIPTION OF THE DRAWINGS

FIGS. 1A-1B are graphs showing baseline MeCP2 protein expression in forebrain and midbrain (FIG. 1A), and olfactory multiciliated cells (FIG. 1B) of MeCP2 knockdown Xenopus laevis and in wild-type control Xenopus laevis.

FIG. 1C is a graph showing baseline MeCP2 RNA expression in the brain and gastrointestinal (GI) tract of MeCP2 knockdown Xenopus laevis and in wild-type control Xenopus laevis.

FIGS. 2A-2C are graphs showing that treatment with vorinostat reverses hyper-acetylation of microtubules and alpha-tubulin in olfactory multi-ciliated cells in Xenopus laevis tadpoles. FIG. 3A is a graph showing that treatment with vorinostat in Xenopus laevis restores normal tongue vertical thickness in MeCP2 knockdown animals.

FIG. 3B is a graph showing that treatment with vorinostat in Xenopus laevis restores normal ib4+ expression in the GI tract in MeCP2 knockdown animals.

FIGS. 4A-4B are a graphs of data from an MeCP2 injected Xenopus laevis tadpole drug screen, showing Seizure Score (FIG. 4A) and Rating Score (FIG. 4B). Key Data: FIG. 4A: Buffer—Injected Control, MecP2—Untreated Control, Trofinetide (70 μg/mL), Trofinetide (140 μg/mL), Sirolimus (30 μM), Vorinostat (25 μM), Vorinostat (50 μM), and Ivermectin (0.1 μM) shown left to right on the graph respectively; FIG. 4B: B. fragilis (10⁶ CFU/mL) and clozapine (5 μM).

FIGS. 5A-5B are graphs showing Seizure Score data for wild-type (FIG. 5A) and MeCP2 knockdown (FIG. 5B) Xenopus laevis tadpoles treated with drug (trofinetide or vorinostat) or vehicle from Day 1 to Day 6, after which they were placed back in medium.

FIGS. 6A-6B are graphs showing that vorinostat improves Xenopus laevis tadpole viability in the MeCP2 knockdown model.

FIG. 7A shows the experimental design for the MeCP2 knockdown mouse studies.

FIGS. 7B-7E show Cumulative Severity Score (FIG. 7B), Fold Change from Initial Weight (FIG. 7C), Elevated Plus Maze (EPM) performance and Enhanced Spatial Novelty Y-Maze performance (FIG. 7D), and Diarrhea Score (FIG. 7E) from the mouse studies.

FIGS. 8A-8B are graphs showing change in animal weight (g) and Cumulative Severity Score following treatment of mice with either trofinetide (FIG. 8A) or vorinostat (FIG. 8B).

FIG. 9A shows a graph showing that oral administration of vorinostat (COG002) in mice treated at 35 days of age, after the onset of severe symptoms, is more effective than trofinetide in reducing the Cumulative Severity Score.

FIGS. 9B-9G are graphs showing change in cumulative mobility score (FIG. 9B), diarrhea score (FIG. 9C), breathing score (FIG. 9D), acetylation of alpha-tubulin in bronchioles (FIG. 9E), acetylation of alpha-tubulin in skeletal muscle (FIG. 9F), and ratio of acetylated alpha-tubulin to bIII-tubulin (FIG. 9G) in mouse studies.

FIG. 10 provides an overview of the mouse neurobehavioral test results as fold change relative to wild type animals.

FIGS. 11A-11B provide data showing that vorinostat treatment restores naïve C57BL6 behavior in anxiety, activity and novelty seeking, using the Elevated Plus Maze test.

FIGS. 12A-12B are graphs showing results from Open Field and Locomotor tests in mice treated with vorinostat or trofinetide.

FIGS. 13A-13B are graphs showing results from Y-Maze Novelty tests in mice treated with vorinostat or trofinetide.

FIG. 14 shows a heat map of plasma cytokine levels in mice treated with vorinostat or trofinetide normalized to wild type animals and shown as Z-score relative to MeCP2 KO animals.

### DETAILED DESCRIPTION

Aspects of the disclosure relate, in part, to methods of ameliorating, reversing, or eliminating symptoms associated with neurodevelopmental disorders such as Rett syndrome (RTT) and CDKL5 deficiency disorder (CDD), which are X-linked developmental brain disorders, in a subject. In some embodiments, methods of ameliorating, reversing, or eliminating symptoms associated with RTT and/or CDD involve administration of vorinostat, ivermectin, trofinetide, a therapeutically effective amount of Bacteroides fragilis and/or a polysaccharide isolated from Bacteroides fragilis to a subject. Some aspects of the disclosure relate to methods of treating neurodevelopmental disorders such as Rett syndrome (RTT) or CDKL5 deficiency disorder (CDD) in a subject, for example, by administration of vorinostat, ivermectin, trofinetide, a therapeutically effective amount of Bacteroides fragilis and/or a polysaccharide isolated from Bacteroides fragilis to the subject.

### Symptoms of Rett Syndrome

Rett syndrome is a rare genetic neurological and developmental disorder that affects the way the brain develops, causing a progressive loss of motor skills and speech. This disorder primarily affects girls. Most babies with Rett syndrome seem to develop normally for the first 6 to 18 months of age, and then lose skills they previously had—such as the ability to crawl, walk, communicate or use their hands. Over time, children with Rett syndrome have increasing problems with the use of muscles that control movement, coordination and communication. Rett syndrome can also cause seizures and intellectual disability. Abnormal hand movements, such as repetitive rubbing or clapping, replace purposeful hand use.

Babies with Rett syndrome typically are born after a normal pregnancy and delivery. Most infants with Rett syndrome seem to grow and behave normally for the first six months. After that, signs and symptoms start to appear.

The most pronounced changes generally occur at 12 to 18 months of age, over a period of weeks or months. Symptoms and their severity can vary greatly from child to child.

Rett syndrome signs and symptoms include, but are not limited to:

Slowed growth. Brain growth slows after birth. Smaller than normal head size (microcephaly) is usually the first sign that a child has Rett syndrome. As children get older, delayed growth in other parts of the body becomes evident.

Loss of normal movement and coordination. The first signs often include reduced hand control and a decreasing ability to crawl or walk normally. At first, this loss of abilities occurs rapidly and then it continues more gradually. Eventually muscles become weak or may become rigid or spastic with abnormal movement and positioning.

Loss of communication abilities. Children with Rett syndrome typically begin to lose the ability to speak, to make eye contact and to communicate in other ways. They may become disinterested in other people, toys and their surroundings. Some children have rapid changes, such as a sudden loss of speech. Over time, children may gradually regain eye contact and develop nonverbal communication skills.

Abnormal hand movements. Children with Rett syndrome typically develop repetitive, purposeless hand movements that may differ for each person. Hand movements may include hand-wringing, squeezing, clapping, tapping or rubbing.

Unusual eye movements. Children with Rett syndrome tend to have unusual eye movements, such as intense staring, blinking, crossed eyes or closing one eye at a time.

Breathing problems. These include breath-holding, abnormally rapid breathing (hyperventilation), forceful exhalation of air or saliva, and swallowing air. These problems tend to occur during waking hours but breathing disturbances such as shallow breathing or periodic breathing can occur during sleep.

Irritability and crying. Children with Rett syndrome may become increasingly agitated and irritable as they get older. Periods of crying or screaming may begin suddenly, for no apparent reason, and last for hours. Some children may experience fears and anxiety.

Anxiety. Anxiety may be assessed using traditional measures and assessments of anxiety and mood behaviors such as RSBQ; Anxiety, Depression, and Mood Scale (ADAMS); and Aberrant Behavior Checklist-Community (ABC-C), which are assessments for people with neurodevelopmental disorders. Anxiety can be assessed using these measures in terms of score profiles, relationship with age and clinical severity, reliability, concurrent validity, and functional implications.

Other abnormal behaviors. These may include, for example, sudden, odd facial expressions and long bouts of laughter, hand licking, and grasping of hair or clothing.

Cognitive disabilities. Loss of skills can be accompanied by a loss of intellectual functioning.

Seizures. Most people who have Rett syndrome experience seizures at some time during their lives. Multiple seizure types may occur and are accompanied by an abnormal electroencephalogram (EEG).

Abnormal curvature of the spine (scoliosis). Scoliosis is common with Rett syndrome. It typically begins between 8 and 11 years of age and increases with age. Surgery may be required if the curvature is severe.

Irregular heartbeat. This is a life-threatening problem for many children and adults with Rett syndrome and can result in sudden death.

Sleep disturbances. Abnormal sleep patterns can include irregular sleep times, falling asleep during the day and being awake at night, or waking in the night with crying or screaming.

Other symptoms. A variety of other symptoms can occur, such as thin, fragile bones prone to factures; small hands and feet that are usually cold; problems with chewing and swallowing; problems with bowel function; and teeth grinding.

### Symptoms of CDKL5 Deficiency Disorder

CDKL5 deficiency disorder is characterized by seizures that begin in infancy, followed by significant delays in many aspects of development.

Seizures in CDKL5 deficiency disorder usually begin within the first 3 months of life and can appear as early as the first week after birth. The types of seizures change with age and may follow a predictable pattern. The most common types are generalized tonic-clonic seizures, which involve a loss of consciousness, muscle rigidity, and convulsions; tonic seizures, which are characterized by abnormal muscle contractions; and epileptic spasms, which involve short episodes of muscle jerks. Seizures occur daily in most people with CDKL5 deficiency disorder, although they can have periods when they are seizure-free.

Development is impaired in children with CDKL5 deficiency disorder. Most have severe intellectual disability and little or no speech. The development of gross motor skills, such as sitting, standing, and walking, is delayed or not achieved. About one-third of affected individuals are able to walk independently. Fine motor skills, such as picking up small objects with the fingers, are also impaired; about half of affected individuals have purposeful use of their hands. Most people with this condition have vision problems (cortical visual impairment).

Other common features of CDKL5 deficiency disorder include repetitive hand movements (stereotypies), such as clapping, hand licking, and hand sucking; teeth grinding (bruxism); disrupted sleep; feeding difficulties; and gastrointestinal problems including constipation and backflow of acidic stomach contents into the esophagus (gastroesophageal reflux). Some affected individuals have episodes of irregular breathing. Distinctive facial features in some people with CDKL5 deficiency disorder include a high and broad forehead, large and deep-set eyes, a well-defined space between the nose and upper lip (philtrum), full lips, widely spaced teeth, and a high roof of the mouth (palate). Other physical differences can also occur, such as an unusually small head size (microcephaly), side-to-side curvature of the spine (scoliosis), and tapered fingers.

CDKL5 deficiency disorder was previously classified as an atypical form of Rett syndrome. These conditions have common features, including seizures, intellectual disability, and other problems with development. However, the signs and symptoms associated with CDKL5 deficiency disorder and its genetic cause are distinct from those of Rett syndrome, and CDKL5 deficiency disorder is now considered a separate condition.

People having CDKL5 deficiency can experience anxiety and mood disturbances. Anxiety may be assessed using traditional measures and assessments of anxiety and mood behaviors such as RSBQ; Anxiety, Depression, and Mood Scale (ADAMS); and Aberrant Behavior Checklist-Community (ABC-C), which are assessments for people with neurodevelopmental disorders. Anxiety can be assessed using these measures in terms of score profiles, relationship with age and clinical severity, reliability, concurrent validity, and functional implications.

### Therapeutic Agents

### Vorinostat

Vorinostat (N′-hydroxy-N-phenyloctanediamide) is a synthetic hydroxamic acid derivative with antineoplastic activity given, for example, in intravenous and oral formulations. Vorinostat, a second generation polar-planar compound, binds to the catalytic domain of the histone deacetylases (HDACs). This allows the hydroxamic moiety to chelate zinc ion located in the catalytic pockets of HDAC, thereby inhibiting deacetylation and leading to an accumulation of both hyperacetylated histones and transcription factors. Hyperacetylation of histone proteins results in the upregulation of the cyclin-dependent kinase p21, followed by G1 arrest. Hyperacetylation of non-histone proteins such as tumor suppressor p53, alpha tubulin, and heat-shock protein 90 produces additional anti-proliferative effects. This agent also induces apoptosis and sensitizes tumor cells to cell death processes. Vorinostat crosses the blood-brain barrier. Vorinostat is approved for use in refractory or relapsed cutaneous T cell lymphoma. Vorinostat is associated with modest rate of minor serum enzyme elevations during therapy but has not been linked to cases of clinically apparent liver injury. Vorinostat is marketed under the name ZOLINZA® for the treatment of cutaneous T cell lymphoma (CTCL). The chemical structure of vorinostat is below:

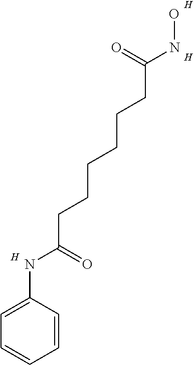

### Ivermectin

Ivermectin is an orally bioavailable macrocyclic lactone derived from Streptomyces avermitilis, with antiparasitic and potential anti-viral activities. Upon administration, ivermectin exerts its anthelmintic effect through binding and activating glutamate-gated chloride channels (GluCls) expressed on nematode neurons and pharyngeal muscle cells. This causes increased permeability of chloride ions, causing a state of hyperpolarization and results in the paralysis and death of the parasite. Ivermectin is typically given as one or two oral doses. Ivermectin therapy has been associated with minor, self-limiting serum aminotransferase elevations and very rare instances of clinically apparent liver injury. The chemical structure of ivermectin is below:

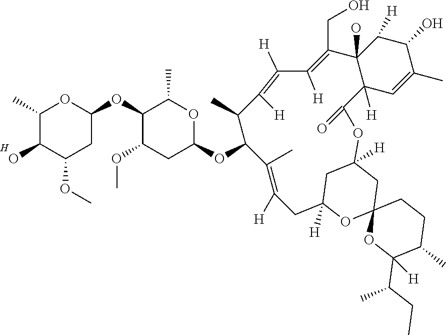

### Trofinetide

Trofinetide (NNZ-2566; (2S)-2-[[(2S)-1-(2-aminoacetyl)-2-methylpyrrolidine-2-carbonyl]amino]pentanedioic acid), a neuroprotective analogue of [glypromate], is a molecule that has a profile suitable for both intravenous infusion and chronic oral delivery. It is currently in development to treat traumatic brain injury. Trofinetide is a modified version of glypromate (GPE), a product of insulin-like growth factor 1 (IGF-1) breakdown in the brain. Both proteins are important for normal functioning of the developing brain, and for responding to disease, stress, and injury in the mature brain. Trofinetide was designed to mimic GPE's biological functions while having better pharmacological properties: easier storage, oral administration, and longer periods in the bloodstream. Some studies have reported the presence of abnormally low brain levels of IGF-1 in several conditions, including Rett syndrome, fragile X syndrome, and brain injury. Restoring the levels of IGF-1 and GPE may help prevent further brain damage in people with these disorders. The chemical structure of trofinetide is below:

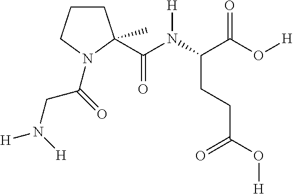 [SMILES: C[C@@]1(C(=O)N[C@@H](CCC(=O)O)C(=O)O)CCCN1C(=O)CN]

Bacteroides fragilis

The B. fragilis group is the most commonly isolated Bacteroidaceae in anaerobic infections, especially those that originate from the gastrointestinal microbiota. B. fragilis is the most prevalent organism in the B. fragilis group, accounting for 41% to 78% of the isolates of the group. These organisms are resistant to penicillin by virtue of production of beta-lactamase, and by other unknown factors. This group was formerly classified as subspecies of B. fragilis (i.e., B. f. ssp. fragilis, B. f. ssp. distasonis, B. f. ssp. ovatus, B. f. ssp. thetaiotaomicron, and B. f. ssp. vulgatus). They have been reclassified into distinct species on the basis of DNA homology studies. B. fragilis (formerly known as B. f. ssp. fragilis) is often recovered from blood, pleural fluid, peritoneal fluid, wounds, and brain abscesses. Although the B. fragilis group is the most common species found in clinical specimens, it is the least common Bacteroides present in fecal microbiota, comprising only 0.5% of the bacteria present in stool. Their pathogenicity partly results from their ability to produce capsular polysaccharide, which is protective against phagocytosis and stimulates abscess formation. The capsule complex of B. fragilis is made up exclusively of Polysaccharide A (PSA) and polysaccharide B (PSB). PSA is a model symbiosis factor, capable of activating T cell-dependent immune responses that can affect both the development and homeostasis of the host immune system.

### Other Therapeutic Agents

The present disclosure contemplates the use of various other agents for the treatment of neurodevelopmental disorders, such as Rett syndrome and/or CDKL5 deficiency disorder, including any one or more agent selected from tyrphostin-AG-1478, sirolimus, vorinostat, panobinostat, entinostat, trichostatin-a, palbociclib, nefazodone, pyrazolanthrone, tamoxifen, wortmannin, azacitidine, indole-3-carbinol, thapsigargin, diphenyleneiodonium, parthenolide, artesunate, chlorpromazine, pifithrin, ivermectin, cycloheximide, fluoxetine, clozapine, ethambutol, selamectin, triclosan, tunicamycin, mifepristone, genistein, and cinobufagin.

In some embodiments, the agent is an agent of Table 1. In some embodiments, the agent is tyrphostin-AG-1478. In some embodiments, the agent is sirolimus. In some embodiments, the agent is vorinostat. In some embodiments, the agent is panobinostat. In some embodiments, the agent is entinostat. In some embodiments, the agent is trichostatin-a. In some embodiments, the agent is palbociclib. In some embodiments, the agent is nefazodone. In some embodiments, the agent is pyrazolanthrone. In some embodiments, the agent is tamoxifen. In some embodiments, the agent is wortmannin. In some embodiments, the agent is azacitidine. In some embodiments, the agent is indole-3-carbinol. In some embodiments, the agent is thapsigargin. In some embodiments, the agent is diphenylenciodonium. In some embodiments, the agent is parthenolide. In some embodiments, the agent is artesunate. In some embodiments, the agent is chlorpromazine. In some embodiments, the agent is pifithrin. In some embodiments, the agent is ivermectin. In some embodiments, the agent is cycloheximide. In some embodiments, the agent is fluoxetine. In some embodiments, the agent is clozapine. In some embodiments, the agent is cthambutol. In some embodiments, the agent is selamectin. In some embodiments, the agent is triclosan. In some embodiments, the agent is tunicamycin. In some embodiments, the agent is mifepristone. In some embodiments, the agent is genistein. In some embodiments, the agent is cinobufagin.

### Pharmaceutical Compositions

In some aspects, the present disclosure provides compositions comprising any of the agents as disclosed herein. In some embodiments, the compositions further comprise a pharmaceutically-acceptable excipient (e.g., carrier, buffer, and/or salt, etc.). A molecule or other substance/agent is considered “pharmaceutically acceptable” if it is approved or approvable by a regulatory agency of the Federal government or a state government or listed in the U.S. Pharmacopcia or other generally recognized pharmacopeia for use in animals, including humans. An excipient may be any inert (inactive), non-toxic agent, administered in combination with an agent provided herein. Non-limiting examples of pharmaceutically-acceptable excipients include water, saline, dextrose, glycerol, ethanol and combinations thereof. The excipient may be selected on the basis of the mode and route of administration, and standard pharmaceutical practice.

General considerations in the formulation and/or manufacture of pharmaceutical agents, such as compositions comprising any of the agents disclosed herein may be found, for example, in Remington's Pharmaceutical Sciences, 18th edition, Mack Publishing Co., Easton, Pa (1990) (incorporated herein by reference in its entirety).

Although the descriptions of pharmaceutical compositions provided in this application are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Therapeutically effective amounts vary, as recognized by those skilled in the art, depending on the route of administration, excipient usage, and co-usage with other active agents. Therapeutically effective amounts depend on the subject, including, for example, the weight, sex and age of the subject as well as the strength of the subject's immune system and/or genetic predisposition. Suitable dosage ranges are readily determinable by one skilled in the art. In some embodiments, an amount of an agent (e.g., vorinostat) is effective to reduce anxiety in a subject, relative to a control. In some embodiments, an amount of an agent (e.g., vorinostat) is effective to improve Rett syndrome scoring of a subject, relative to a control. In some embodiments, an amount of an agent (e.g., vorinostat) is effective to decrease inflammation in a subject, relative to a control. In some embodiments, an amount of an agent (e.g., vorinostat) is effective to reduce, inhibit, or reverse ciliary dysfunction in a subject, relative to a control. Motile ciliary dysfunction may lead to pulmonary infections and worsening respiratory function due to poor mucociliary clearance of pathogens and debris in the airways, brain ventricle dysregulation of cerebrospinal fluid flow, and oviduct motility. Primary ciliary dysfunction impacts mechanosensation across many organs and tissues, including bone, kidney ducts, pancreas, and vasculature/heart development.

### Therapies

Any of the agents or compositions disclosed herein may be administered to a subject to treat (e.g., ameliorate, reduce or alleviate) a symptom of a neurodevelopmental disorder such as Rett syndrome or CDKL5 deficiency disorder. Any of the agents or compositions disclosed herein may be administered to a subject to treat Rett syndrome or CDKL5 deficiency disorder.

In some embodiments, administration of any of the agents or compositions described herein (e.g., vorinostat, ivermectin, trofinetide, a therapeutically effective amount of Bacteroides fragilis and/or a polysaccharide isolated from Bacteroides fragilis) may be useful in treating existing symptoms, preventing additional symptoms, ameliorating or preventing the underlying causes of symptoms, preventing or reversing causes of symptoms associated with Rett syndrome or CDKL5 deficiency. Administration of any of the agents or compositions described herein (e.g., vorinostat, ivermectin, trofinetide, a therapeutically effective amount of Bacteroides fragilis and/or a polysaccharide isolated from Bacteroides fragilis) provide a beneficial result on a subject with symptoms from a disorder (e.g., Rett syndrome), or a subject with the potential to develop symptoms from a disorder. Furthermore, “treatment” may be defined as the application or administration of an agent (e.g., therapeutic agent or a therapeutic composition) to a subject, or an isolated tissue or cell line from a subject, who may have Rett syndrome or CDKL5 deficiency, a symptom of these diseases, with the purpose to cure, heal, alleviate, relieve, alter, remedy, ameliorate, improve or affect the disease, the symptoms of disease or the predisposition toward disease.

A subject herein refers to a human. The subject, in some embodiments, has been diagnosed with Rett syndrome or CDKL5 deficiency disorder. Thus, in some embodiments, the subject is a Rett syndrome patient or CDKL5 deficiency disorder patient under the care of a physician or other medical professional. In other embodiments, the subject exhibits symptoms of Rett syndrome or CDKL5 deficiency disorder but has not been diagnosed with Rett syndrome or CDKL5 deficiency disorder.

An effective amount of any agent or composition described herein refers to an administered amount or concentration of an agent or composition that is necessary and sufficient to treat (e.g., ameliorate, reduce or alleviate) one or more symptoms of a neurodevelopmental disorder such as Rett syndrome or CDKL5 deficiency, e.g., in a subject. In some embodiments, an effective amount of any agent or composition described herein refers to an administered amount or concentration of an agent or composition that is necessary and sufficient to elicit a biological response, e.g., in a subject.

An effective amount of vorinostat may be an amount of vorinostat that is lower than the amount of vorinostat approved by the U.S. Food and Drug Administration (FDA) for the treatment of cutaneous T cell lymphoma (CTCL). Thus, in some embodiments, an effective amount of vorinostat is lower than the amount and dosage as described in Mann B. S. et. al. FDA approval summary: Vorinostat for treatment of advanced primary cutaneous T-cell lymphoma. Oncologist. 2007; 12:1247-52. In some embodiments, an effective amount of vorinostat is 20-1000 mg per day, 20-500 mg per day, 20-200 mg per day, 100-1000 mg per day, 100-500 mg per day, 200-500 mg per day, or 200-400 mg per day.

An effective amount of trofinetide may be as described in Glaze, D. et. al. Double-blind, randomized, placebo-controlled study of trofinetide in pediatric Rett syndrome. Neurology. April 16, 2019; 92 (16). In some embodiments, an effective amount of trofinetide is 20-1000 mg/kg, 20-500 mg/kg, 20-200 mg/kg, 100-1000 mg/kg, 100-500 mg/kg, 200-500 mg/kg, or 200-400 mg/kg.

An effective amount of ivermectin may be as described in Shirazi, F. M. et al., Repurposing the drug, ivermectin, in COVID-19: toxicological points of view. European Journal of Medical Research volume 27, Article number: 21 (2022).; or Kamgno J, et al. Adverse systemic reactions to treatment of onchocerciasis with ivermectin at normal and high doses given annually or three-monthly. Trans R Soc Trop Med Hyg. 2004; 98 (8): 496-504. In some embodiments, an effective amount of ivermectin is 20-1000 mg per day, 20-500 mg per day, 20-200 mg per day, 100-1000 mg per day, 100-500 mg per day, 200-500 mg per day, or 200-400 mg per day.

An effective amount of Bacteroides fragilis may be as described in Zhang, W. et. al., Bacteroides fragilis Protects Against Antibiotic-Associated Diarrhea in Rats by Modulating Intestinal Defenses. Front. Immunol., 9 May 2018. doi.org/10.3389/fimmu.2018.01040. In some embodiments, an effective amount of Bacteroides fragilis is at least 10⁴, 10⁵, 10⁶, 10⁷, 10⁸, or 109 colony-forming units (CFU).

An effective amount of a polysaccharide isolated from Bacteroides fragilis may be as described in Zheng, L. et. al., Capsular Polysaccharide From Bacteroides fragilis Protects Against Ulcerative Colitis in an Undegraded Form. Front. Pharmacol., 7 Dec. 2020 doi.org/10.3389/fphar.2020.570476. In some embodiments, an effective amount of a polysaccharide isolated from Bacteroides fragilis is 1-100 mg/kg, 1-50 mg/kg, 2-50 mg/kg, 2-25 mg/kg, 2-10 mg/kg, 2-5 mg/kg, or 5-50 mg/kg.

Suitable routes of administration include, without limitation, intravenous, intrathecal, intranasal, aerosol, oral (e.g., sublingual), intramuscular, subcutaneous, and inhalation. In some embodiments, an agent of the disclosure is administered intravenously, subcutaneous, intramuscularly or intranasally. In some embodiments, an agent of the disclosure is delivered to the lung, for example, via aerosol, nebulizer, or tracheal wash. Other routes of administration are contemplated herein. The administration route of an agent of the disclosure can be changed depending on a number of factors, including the pathogen and/or mechanism of pathogenesis. The route of administration of the compositions provided herein may vary depending on the specific agents (e.g., vorinostat, chemical compound, a programmable nuclease, or a small molecule).

In some embodiments, administration of any of the agents or compositions described herein (e.g., vorinostat, ivermectin, trofinetide, a therapeutically effective amount of Bacteroides fragilis and/or a polysaccharide isolated from Bacteroides fragilis) to a subject reduces the severity of one or more symptoms of Rett Syndrome by at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to a control (e.g., baseline severity of the one or more symptoms in the subject prior to administration of the agent or composition; or a subject who has not been administered the agent or composition).

In some embodiments, administration of any of the agents or compositions described herein (e.g., vorinostat, ivermectin, trofinetide, a therapeutically effective amount of Bacteroides fragilis and/or a polysaccharide isolated from Bacteroides fragilis) to a subject reduces the severity of one or more symptoms of CDKL5 deficiency by at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to a control (e.g., baseline severity of the one or more symptoms in the subject prior to administration of the agent or composition; or a subject who has not been administered the agent or composition).

For example, in some embodiments, administration of any of the agents or compositions described herein (e.g., vorinostat, ivermectin, trofinetide, a therapeutically effective amount of Bacteroides fragilis and/or a polysaccharide isolated from Bacteroides fragilis) to a subject reduces anxiety in the subject by at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to a control (e.g., baseline severity of the one or more symptoms in the subject prior to administration of the agent or composition; or a subject who has not been administered the agent or composition). In some embodiments, the level of anxiety in the subject can be assessed using a traditional measure of anxiety (e.g., RSBQ; Anxiety, Depression, and Mood Scale (ADAMS); and Aberrant Behavior Checklist-Community (ABC-C)). In some embodiments, a measure of anxiety is assessed using a method as described in Barnes, K. et. al., Anxiety-like behavior in Rett syndrome: characteristics and assessment by anxiety scales. J Neurodev Disord. 2015; 7 (1): 30.

In some embodiments, administration of any of the agents or compositions described herein (e.g., vorinostat, ivermectin, trofinetide, a therapeutically effective amount of Bacteroides fragilis and/or a polysaccharide isolated from Bacteroides fragilis) to a subject improves a Rett syndrome score in the subject by at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to a control (e.g., baseline severity of the one or more symptoms in the subject prior to administration of the agent or composition; or a subject who has not been administered the agent or composition). In some embodiments, a Rett syndrome score is as described in Fabio, A. R. et. al., The GAIRS Checklist: a useful global assessment tool in patients with Rett syndrome. Orphanet Journal of Rare Diseases, volume 17, Article 116 (2022); or Downs, J. et. al., Validating the Rett Syndrome Gross Motor Scale. PLOS One. 2016; 11 (1): c0147555.

In some embodiments, administration of any of the agents or compositions described herein (e.g., vorinostat, ivermectin, trofinetide, a therapeutically effective amount of Bacteroides fragilis and/or a polysaccharide isolated from Bacteroides fragilis) to a subject reduces inflammation (e.g., local inflammation in the brain, or systemic inflammation) in the subject by at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to a control (e.g., baseline severity of the one or more symptoms in the subject prior to administration of the agent or composition; or a subject who has not been administered the agent or composition). In some embodiments, a subject has chronic inflammation. In some embodiments, the level of inflammation in the subject can be assessed using a traditional measure of inflammation. In some embodiments, the level of inflammation in the subject can be assessed by measuring the levels of inflammatory biomarkers (e.g., VEGF, ICAM, VCAM, SAA, or CRP) and/or cytokines (e.g., FN-a, IFN-p, IFN-y, IL-1a, IL-1 IP, IL-1 receptor antagonist, IL-2, IL-4, IL 5, IL-6, soluble IL-6 receptor, IL-7, IL-8, IL-9, IL-10, IL-12, IL-13, IL-15, IL-17, IL-23, or IL-27).

In some embodiments, administration of any of the agents or compositions described herein (e.g., vorinostat, ivermectin, trofinetide, a therapeutically effective amount of Bacteroides fragilis and/or a polysaccharide isolated from Bacteroides fragilis) to a subject reduce, inhibit, or reverse ciliary dysfunction (or ciliogenesis) in cells of the subject by at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to a control (e.g., baseline severity of the one or more symptoms in the subject prior to administration of the agent or composition; or a subject who has not been administered the agent or composition). In some embodiments, a measure of ciliary dysfunction is assessed using a method as described in Frasca, A. et. al., MECP2 mutations affect ciliogenesis: a novel perspective for Rett syndrome and related disorders. EMBO Mol Med. 2020 Jun. 8; 12 (6): c10270.

In some embodiments, administration of any of the agents or compositions described herein (e.g., vorinostat, ivermectin, trofinetide, a therapeutically effective amount of Bacteroides fragilis and/or a polysaccharide isolated from Bacteroides fragilis) to a subject reduce or inhibit seizures in the subject by at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 150%, at least 200%, at least 250%, at least 300%, at least 350%, at least 400%, at least 500%, or at least 1000% relative to a control (e.g., baseline severity of the one or more symptoms in the subject prior to administration of the agent or composition; or a subject who has not been administered the agent or composition). In some embodiments, the intensity of seizures are reduced. In some embodiments, the number of seizures (e.g., the number of seizures in a given week or month) are reduced.

The compositions herein may be administered as a single dose or as multiple doses (e.g., a booster dose or multiple booster doses). In certain embodiments, when multiple doses are administered to a subject, any two doses of the multiple doses include different or substantially the same amounts of an agent described in this application. Dosage forms may be administered at a variety of frequencies. In certain embodiments, when multiple doses are administered to a subject, the frequency of administering the multiple doses to the subject is three doses a day, two doses a day, one dose a day, one dose every other day, one dose every third day, one dose every week, one dose every two weeks, one dose every three weeks, or one dose every four weeks, or less frequent than every four weeks. In certain embodiments, the frequency of administering the multiple doses to the subject is one dose per day. In certain embodiments, the frequency of administering the multiple doses to the subject is two doses per day. In certain embodiments, the frequency of administering the multiple doses to the subject is three doses per day. In certain embodiments, when multiple doses are administered to a subject, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject. In certain embodiments, the duration between the first dose and last dose of the multiple doses is three months, six months, or one year. In certain embodiments, the duration between the first dose and last dose of the multiple doses is the lifetime of the subject. In some embodiments, dose ranging studies can be conducted to establish optimal therapeutic or effective amounts of the component(s) (e.g., proteins or peptides) to be present in dosage forms.

In some embodiments, a subject undergoing treatment has been diagnosed with Rett syndrome. In some embodiments, the subject has been diagnosed with CDKL5 deficiency disorder.

In some embodiments, the vorinostat is formulated with hydroxypropyl B-cyclodextrin.

In some embodiments, the amount of vorinostat is effective to reduce anxiety in the subject, relative to a control. In some embodiments, the amount of vorinostat is effective to improve Rett syndrome scoring of the subject, relative to a control. In some embodiments, the amount of vorinostat is effective to decrease inflammation (e.g., inflammation in the gastrointestinal tract) in the subject, relative to a control. In some embodiments, the amount of vorinostat is effective reduce, inhibit, or reverse ciliary dysfunction in the subject, relative to a control.

In some embodiments, amount of vorinostat is lower than the amount of vorinostat approved by the U.S. Food and Drug Administration (FDA) for the treatment of cutaneous T cell lymphoma (CTCL).

In some embodiments, the amount of ivermectin is effective to reduce or inhibit seizures in the subject.

In some embodiments, the Bacteroides fragilis is Bacteroides fragilis 9343.

In some embodiments, the polysaccharide is polysaccharide A (PSA) or polysaccharide B (PSB).

### Combination Therapies

Any combination of two or more of the agents provided herein may be administered to a subject to treat a symptom of Rett syndrome or CDKL5 deficiency disorder.

In some embodiments, more than one agents associated with the disclosure is administered to a subject. In some embodiments, the agents are administered concomitantly. In other embodiments, the agents are not administered concomitantly. In some embodiments, the first agent is not administered within 1 month, 1 week, 6 days, 5, days, 4 days, 3 days, 2 days, 1 day, 12 hour, 6 hours, 5 hours, 4 hours, 3 hours, 2 hours, or 1 hour of the second agent. The term “concomitantly” refers to administering two or more agents to a subject in a manner that is correlated in time, preferably sufficiently correlated in time such that a first agent has an effect on a second agent, such as increasing the efficacy of the second agent, preferably the two or more agents are administered in combination. In some instance, a second agent has an effect on a first agent, such as regulating the efficacy of the first composition. In some embodiments, concomitant administration may encompass administration of two or more agents within a specified period of time. In some embodiments, the two or more agents are administered within 1 month, within 1 week, within 1 day, or within 1 hour. In some embodiments, concomitant administration encompasses simultaneous administration of two or more agents. In some embodiments, when two or more agents are not administered concomitantly, there is little to no effect of the first agent on the second agent.

In some embodiments, a method of treating a symptom of Rett syndrome or CDKL5 deficiency disorder in a subject in need thereof comprises administering to the subject a therapeutically effective amount of vorinostat and ivermectin.

In some embodiments, a method of treating a symptom of Rett syndrome or CDKL5 deficiency disorder in a subject in need thereof comprises administering to the subject a therapeutically effective amount of vorinostat and trofinetide.

In some embodiments, a method of treating a symptom of Rett syndrome or CDKL5 deficiency disorder in a subject in need thereof comprises administering to the subject a therapeutically effective amount of vorinostat and Bacteroides fragilis or a polysaccharide isolated from Bacteroides fragilis.

In some embodiments, a method of treating a symptom of Rett syndrome or CDKL5 deficiency disorder in a subject in need thereof comprises administering to the subject a therapeutically effective amount of ivermectin and trofinetide.

In some embodiments, a method of treating a symptom of Rett syndrome or CDKL5 deficiency disorder in a subject in need thereof comprises administering to the subject a therapeutically effective amount of ivermectin and Bacteroides fragilis or a polysaccharide isolated from Bacteroides fragilis.

In some embodiments, a method of treating a symptom of Rett syndrome or CDKL5 deficiency disorder in a subject in need thereof comprises administering to the subject a therapeutically effective amount of trofinetide and Bacteroides fragilis or a polysaccharide isolated from Bacteroides fragilis.

### EXAMPLES

### Example 1

Xenopus laevis tadpoles having an MeCP2 knockdown were generated using CRISPR-Cas9 technology. Xenopus embryos were fertilized and maintained at 14° C. until the 4-cell stage. Each cell was injected with ˜2 nL ribonucleoprotein (a mixture of sgRNAs targeting the MeCP2 gene and Cas9 enzyme). No adverse effects to development were observed, as all embryos developed to the swimming tadpole stages (Nieuwkoop-Faber stages 45-50) with no apparent loss of viability or morphological defect.

Swimming tadpoles with MeCP2 knock down (“Rett” tadpoles) exhibited a broad range of abnormal behavior compared to wild-type control tadpoles (“Control”) including darting motions and rapid repetitive swimming in tight circles that was analogous to the repetitive motions observed in human Rett patients; and behavior that has been shown to correspond to seizures in prior Xenopus studies.

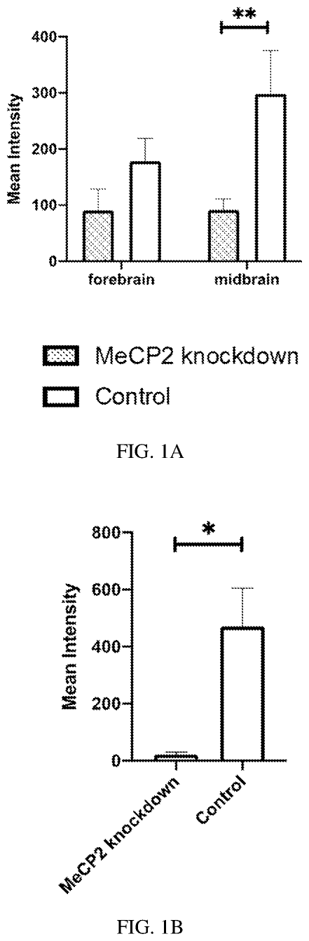

Baseline MeCP2 expression was evaluated in Stage 46 Xenopus laevis forebrain and midbrain in MeCP2 knockdown animals and in wild-type control animals. See FIGS. 1A-1C. MeCP2 protein levels (observed using immunohistochemistry) were reduced in the forebrain and midbrain (FIG. 1A) and olfactory multiciliated cells (MCC) (FIG. 1B) of the MeCP2 knockdown animals. MeCP2 RNA expression levels (observed using RNAscope) in the brain and gastrointestinal (GI) tract of the MeCP2 knockdown animals.

### Example 2

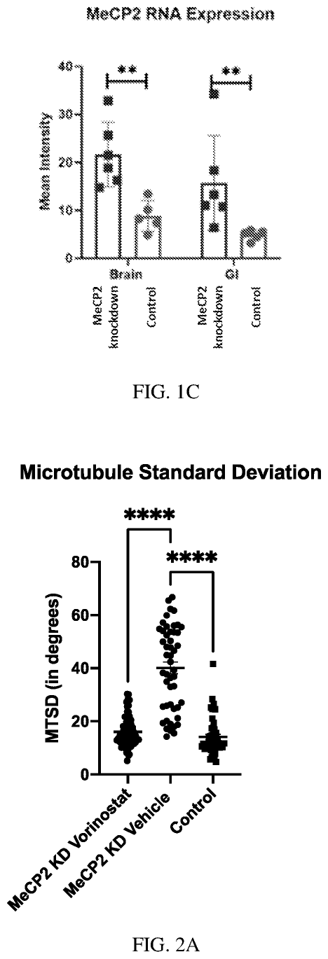

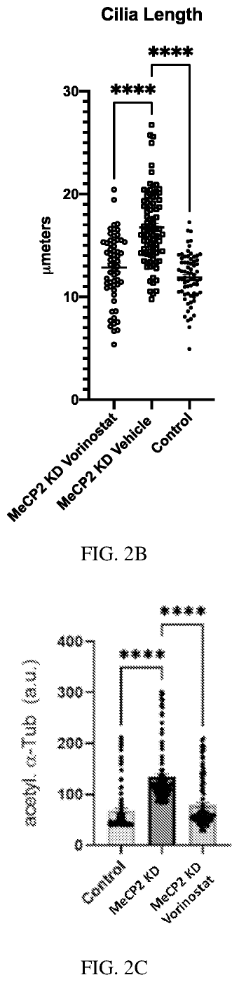

Treatment with vorinostat reversed hyper-acetylation in olfactory multi-ciliated cells in the Xenopus laevis tadpoles with knockdown levels of MeCP2 that were generated in Example 1. See FIGS. 2A-2C.

Specifically, tadpole tissues were assessed for alpha-tubulin acetylation and bidirectional shifts in acetylation patterns were observed depending on the tissue type. For example, alpha-tubulin was significantly hypoacetylated in neurons in sections of the midbrain and in olfactory bulb multi-ciliated cells that showed MeCP2 knockdown, whereas in the gastrointestinal (GI) tract, a-tubulin was hyperacetylated. Treatment of MeCP2 knockdown animals with vorinostat reversed that hyperacetylation in the GI tract (FIG. 2C). Cilia in the nostrils, which are optically accessible for live imaging, were also denser in MeCP2 knockdown animals, and higher resolution imaging revealed that the cilia were longer and misaligned on multiciliated cells compared to controls. Treatment of MeCP2 knockdown animals with vorinostat reversed those effects related to microtubules and cilia length (FIGS. 2A-2B).

### Example 3

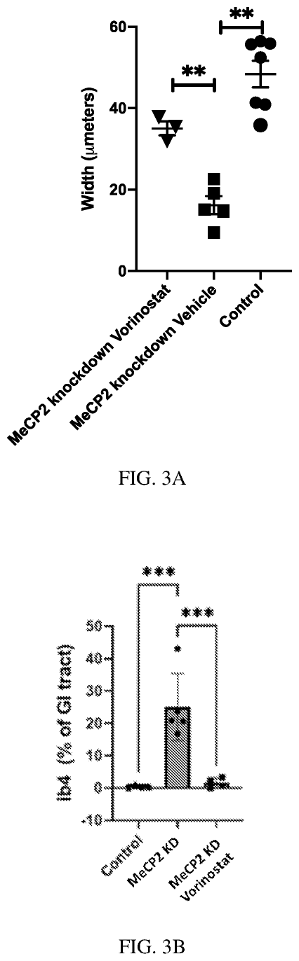

Treatment with vorinostat in Xenopus laevis restored normal tongue thickness in MeCP2 knockdown animals. See FIG. 3A.

Treatment with vorinostat in Xenopus laevis also had impacts on GI tract inflammation and nociceptive innervation. The role of MeCP2 knockdown in GI tract inflammation and nociceptive innervation by staining for isolectin B4-positive (ib4+) cells. Tadpoles having MeCP2 knockdown exhibited significant ib4+ expression; treatment with vorinostat reversed that ib4+ expression. See FIG. 3B.

### Example 4

Seizure Score and Rating Score were assessed in a drug screen using MeCP2 injected

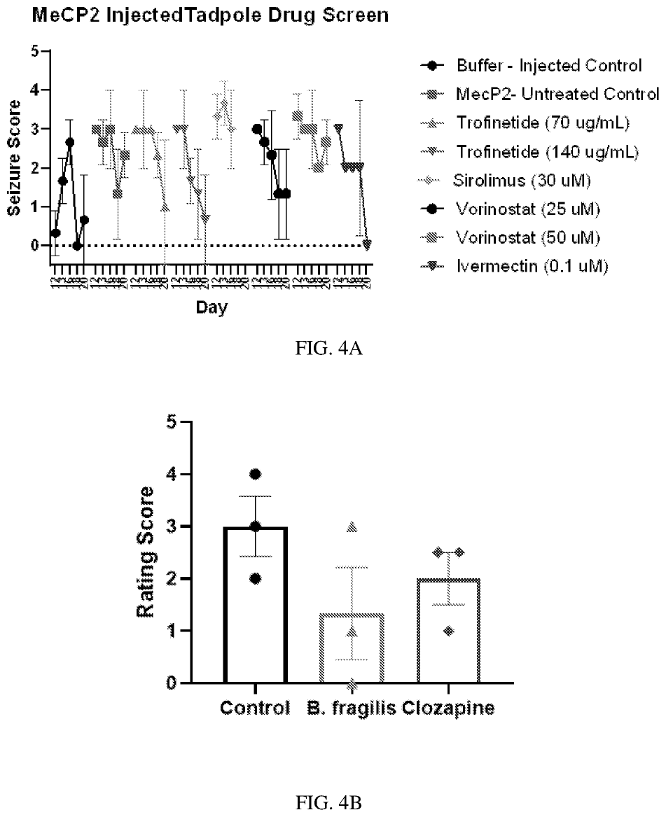

Xenopus laevis tadpoles See FIGS. 4A-4B.

### Example 5

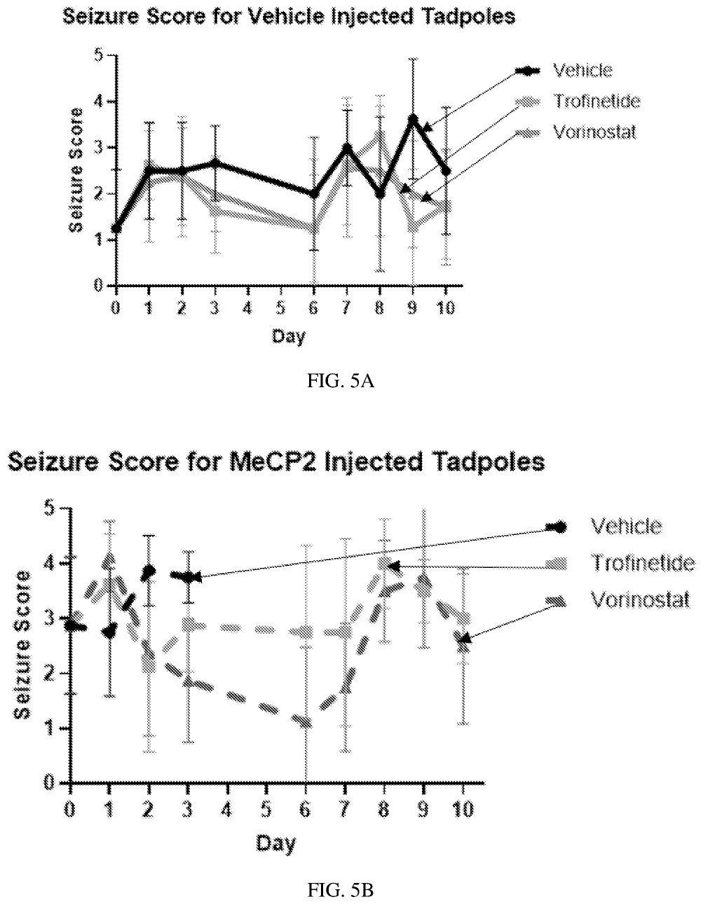

Seizure Score data for wild-type and MeCP2 knockdown was collected for Xenopus laevis tadpoles treated with drug (trofinetide or vorinostat) or vehicle from Day 1 to Day 6, after which they were placed back in medium. See FIGS. 5A-5B. Trofinetide and vorinostat reduced seizures in MeCP2 knockdown animals, relative to animals treated with vehicle.

### Example 6

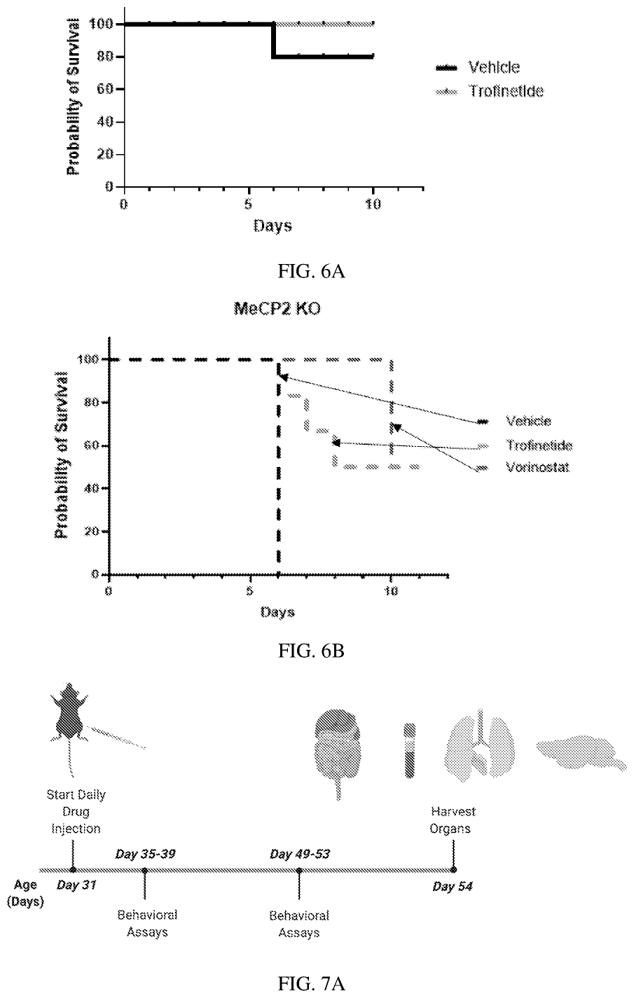

Vorinostat was shown to improve Xenopus laevis tadpole viability in the MeCP2 knockdown model, relative to wild-type Xenopus laevis tadpole. See FIGS. 6A-6B. 100% of MeCP2 knockdown animals treated with vorinostat survived until Day 10; over 50% of MeCP2 knockdown animals treated with trofinetide survived until Day 10. Conversely, none of the MeCP2 knockdown animals treated with vehicle survived beyond Day 6.

### Example 7

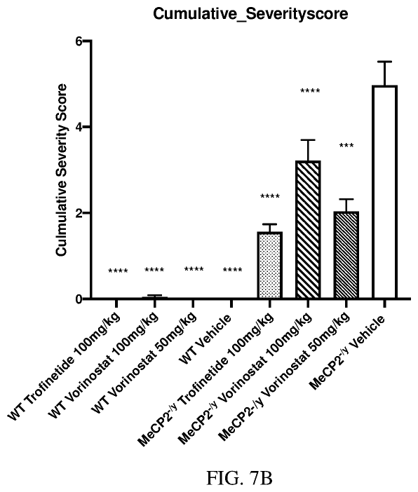

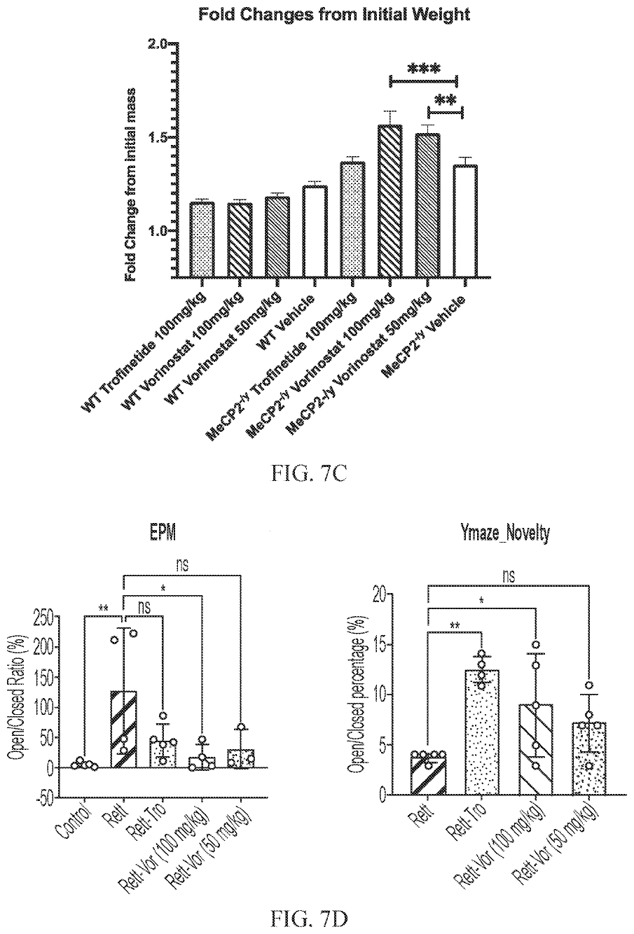

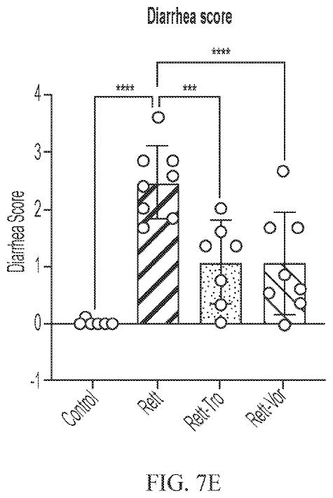

MeCP2 knockdown mouse studies were performed and Cumulative Severity Score and Fold Change from Initial Weight were assessed. See FIGS. 7A-7C.

Wild-type and MeCP2-knockdown (MeCP2^{-/Y}) mice (a Rett mouse model) were treated starting at day 31 post-partum (p31) via daily intraperitoneal (i.p.) administration of trofinetide (100 mg/kg) or vorinostat (50 mg/kg) until day 51 of age (FIG. 7A). Behavorial assays were performed at days 35-39 and days 49-53, before sacrificing the mice and harvesting organs at day 54.

The MeCP2-knockdown mice had cumulative severity scores (as assessed using the severity score described in Guy, J., Gan, J., Selfridge, J., Cobb, S. & Bird, A. Reversal of Neurological Defects in a Mouse Model of Rett Syndrome. Science 315, 1143-1147 (2007)) that were significantly higher than wild-type mice. Treatment of the MeCP2-knockdown mice resulted in significant amelioration of multiple disease-related parameters, including diarrhea and motor function, measured by a reduction in the severity scores (FIG. 7B).

Treatment of the MeCP2-knockdown mice with vorinostat also resulted in improved neurological function relative to vehicle-treated MeCP2-knockdown mice, as determined by measuring elevated plus maze (EPM) performance and enhanced spatial novelty Y-maze performance (FIG. 7D). Additionally, treatment of MeCP2-knockdown mice with vorinostat resulted in an improved diarrhea score (FIG. 7E) relative to vehicle-treated MeCP2-knockdown mice, demonstrating the effects of vorinostat on the function of the GI tract.

### Example 8

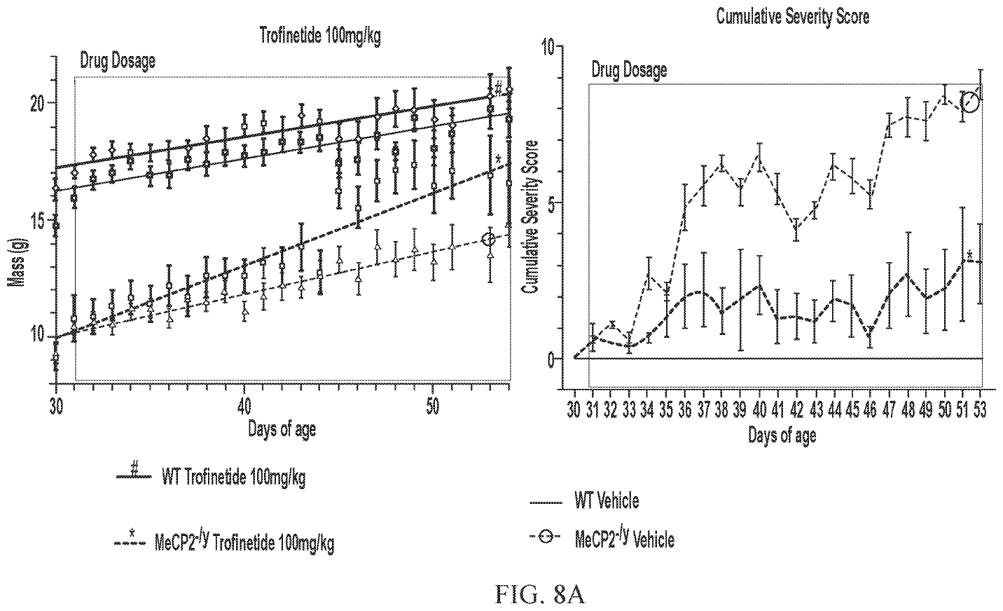

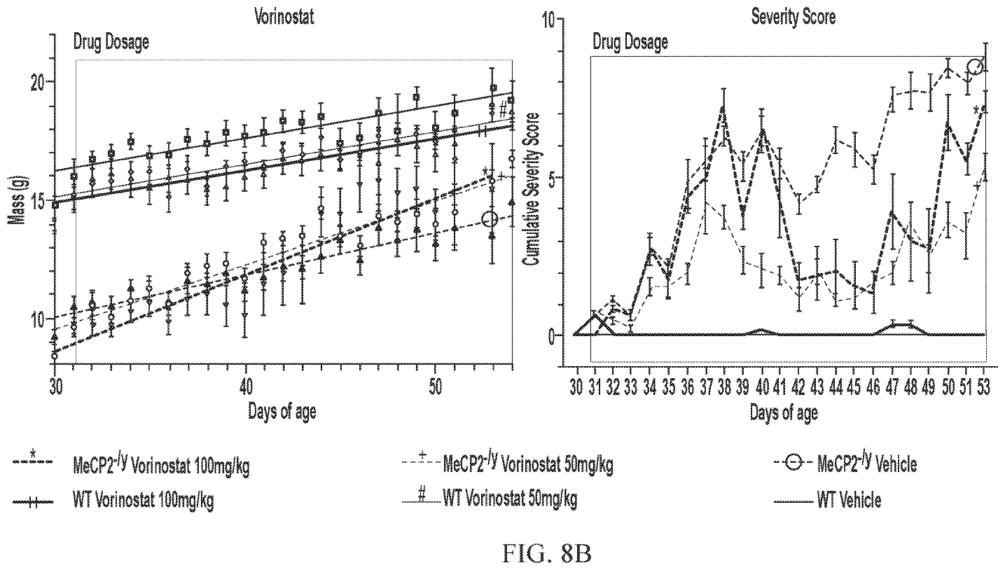

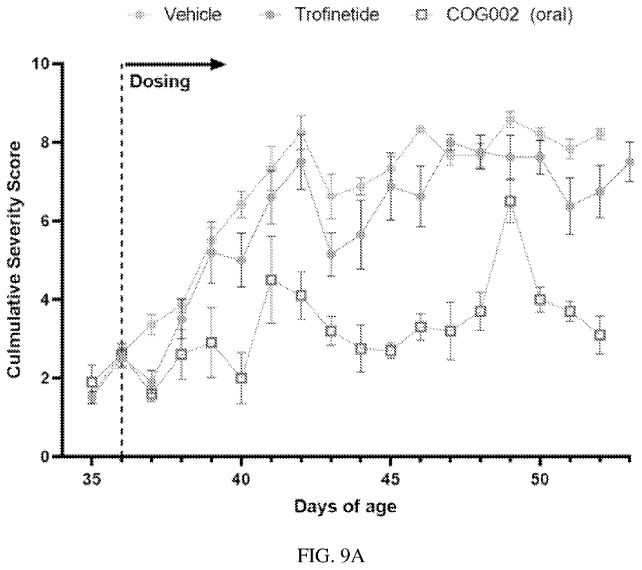

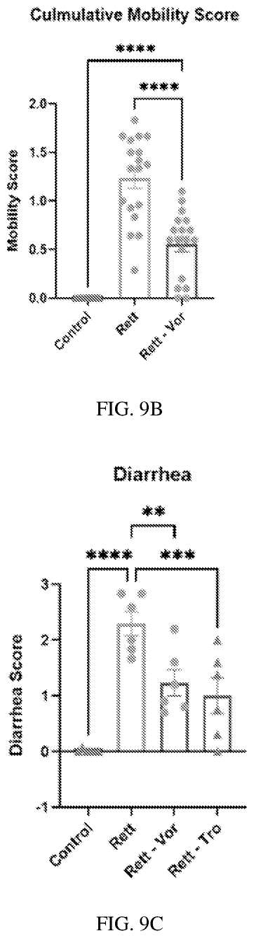

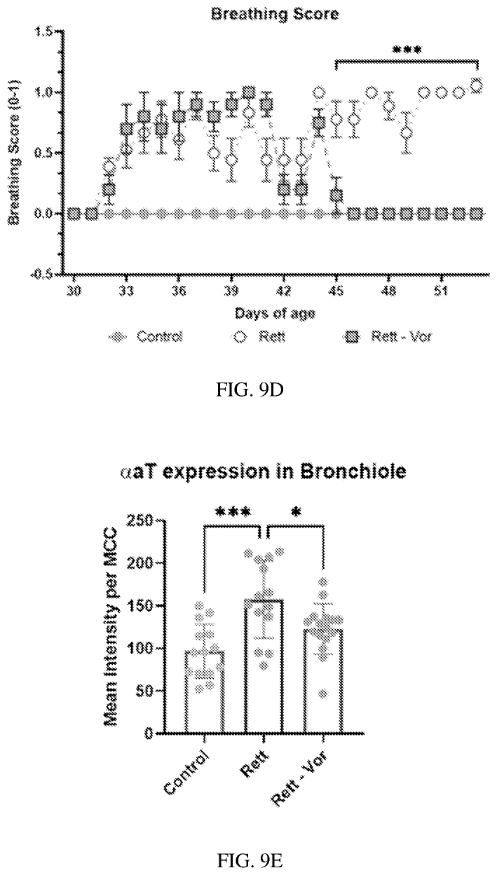

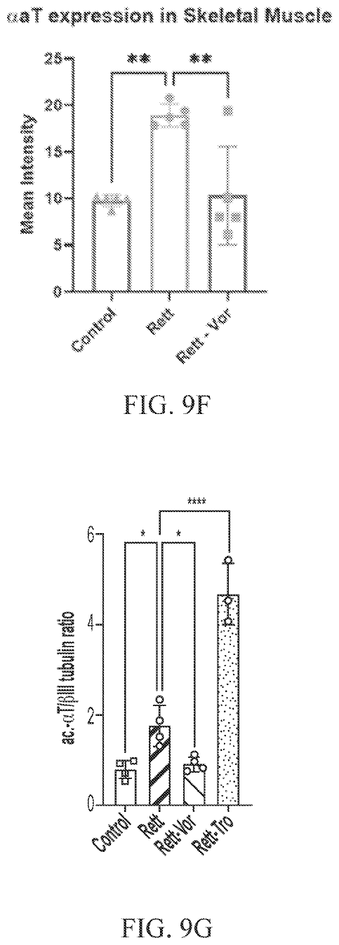

Mice were treated with either trofinetide or vorinostat, and change in animal weight (g) and Cumulative Severity Score were assessed. See FIGS. 8A-8B. Oral administration of vorinostat (COG002) in mice treated at 35 days of age, after the onset of severe symptoms, was more effective than trofinetide in reducing the Cumulative Severity Score. See FIG. 9.

MeCP2-knockdown (MeCP2-/Y) mice were dosed approximately one week after the onset of symptoms with an oral formulation of vorinostat. Oral vorinostat (50 mg/kg) prevented significant worsening of the symptom severity score (FIG. 9A), ameliorated weight gain, and increased performance in EPM and Y mazes. 100% of MeCP2-knockdown animals treated with oral vorinostat showed survival after 3 weeks of treatment, whereas about 60% of trofinetide-treated MeCP2-knockdown animals.

Additionally, the overall mobility scores (based on mobility, gait, and hindlimb clasping) and the diarrhea scores were improved for MeCP2-knockdown animals treated with vorinostat and trofinetide, relative to vehicle-treated MeCP2-knockdown animals (FIG. 9B and 9C, respectively). Breathing also was significantly improved following vorinostat treatment compared to trofinetide and vehicle treatments (FIG. 9D). There was also an overall increase of hyperacetylated a-tubulin in bronchioles of MeCP2-Vy animals that vorinostat restored (FIG. 9E). Finally, vorinostat treatment rescued the disrupted a-tubulin acetylation observed in femoral muscle sections (FIG. 9F). Increased acetylation due to the loss of MeCP2 may play a role in muscle function as hyperacetylation has been shown to increase stiffness and resistance in striatal muscles in vitro.

Initial histological analysis of the colon indicated a greater degree of vacuolization and heightened neutrophil infiltrate in MeCP2-knockdown animals, which vorinostat and trofinetide reversed. Staining of these sections for bIII-and acetylated alpha-tubulin also revealed that acetylated a-tubulin is significantly more colocalized with neuronal staining in the colon in MeCP2-knockdon animals, and that this can be normalized by treatment with either vorinostat or trofinetide. Vorinostat also normalized the ratio of acetylated alpha-tubulin to bIII-tubulin (FIG. 9G).

Collectively, these data demonstrate that vorinostat is capable of ameliorating symptoms of Rett syndrome following the onset of said symptoms.

### Example 9

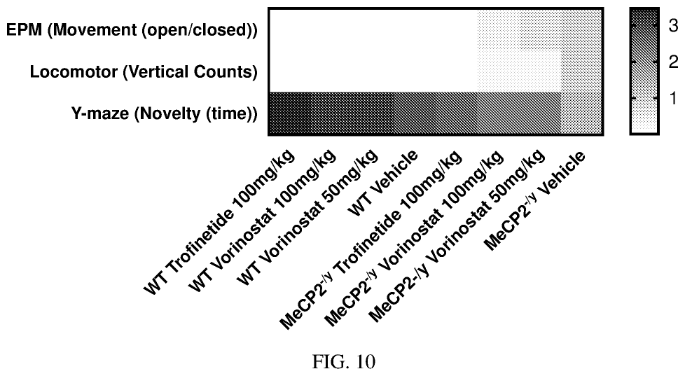

Mouse neurobehavioral tests were performed, including EPM, locomotor, and Y-maze. The test results are shown in FIG. 10 as fold changes relative to wild type animals.

### Example 10

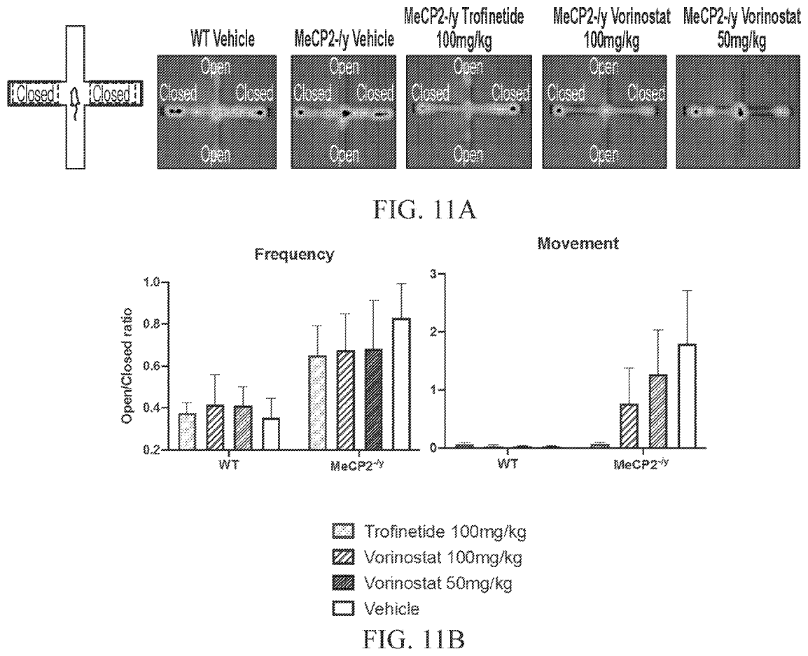

Naïve C57BL6 mice were treated with vorinostat, which restored behavior in anxiety, activity and novelty seeking, using the Elevated Plus Maze test. See FIGS. 11A-11B.

### Example 11

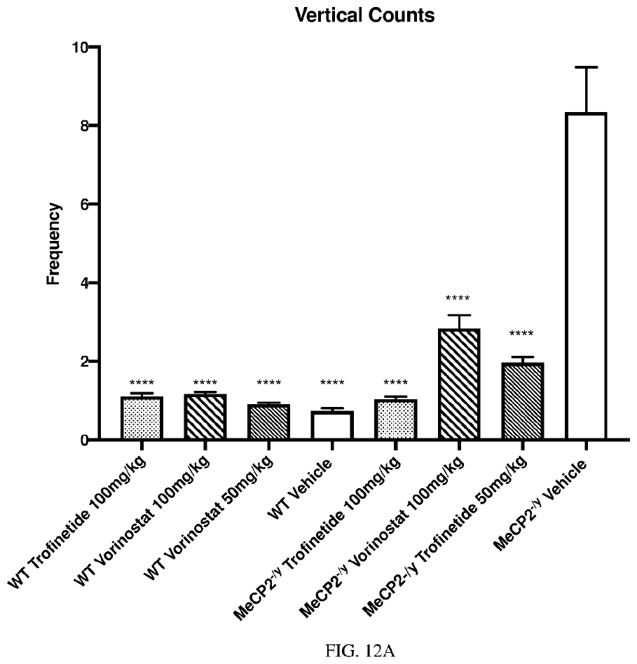

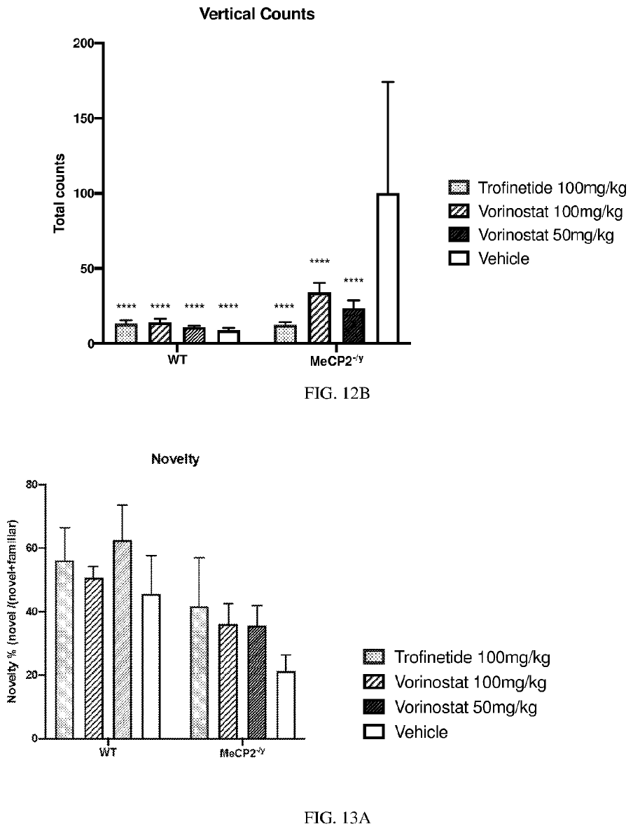

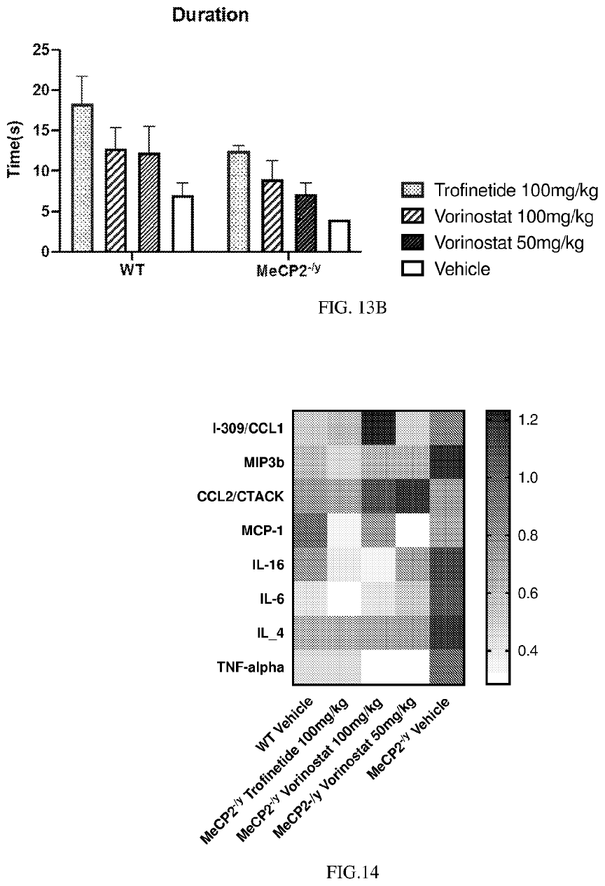

Open Field and Locomotor tests were performed in mice treated with vorinostat or trofinetide. Results are shown in FIGS. 12A-12B. Y-Maze tests were also performed in mice treated with vorinostat or trofinetide. Results are shown in FIG. 13A (novelty) and FIG. 13B (duration). FIG. 14 shows a heat map of plasma cytokine levels in mice treated with vorinostat or trofinetide normalized to wild type animals and shown as Z-score relative to MeCP2 KO animals.

### Example 12

To identify drugs for use in treating symptoms associated with neurodevelopmental disorders, such as Rett syndrome and CDKL5 deficiency disorder, probabilistic network maps were generated for each drug in an aggregate 19,800+compound dataset. By inputting and comparing gene microarray data from MeCP2 knockdown tadpoles compared to wild-type controls, sets of differentially expressed genes were identified within the gene-gene interaction network with a p-value <0.05 and a log2 fold change between 0.7 and 3.1 in 0.3-fold-change increments. For each fold-change, a ranked list of compounds predicted to induce reversal of transcriptome-wide changes of: 1) single gene states using a cross-correlation score, 2) single gene states using a cross-entropy score, and 3) an overall gene network state score corresponding to the probability of involvement of the drug nodes in the identified subnetwork. These drug lists were combined to identify drugs that were ranked by being most consistently predicted to reverse the Rett transcriptome-wide changes and robust across the computational settings.

The following compounds of Table 1 were computationally predicted for use in treating symptoms associated with neurodevelopmental disorders, such as Rett syndrome and CDKL5 deficiency disorder. The prediction score, analogous to a distance from a healthy target state, is shown on the right. A lower score indicates better predicted efficacy.

TABLE 1
Computationally Predicted Drugs for
Treating Neurodevelopmental Disorders

| Drug | Score |
|---|---|
| tyrphostin-AG-1478 | 20.5 |
| sirolimus | 28.875 |
| vorinostat | 33.625 |
| panobinostat | 37.875 |
| entinostat | 42.75 |
| trichostatin-a | 49 |
| palbociclib | 56.125 |
| nefazodone | 57.75 |
| pyrazolanthrone | 60.875 |
| tamoxifen | 65.75 |
| wortmannin | 73 |
| azacitidine | 76.625 |
| indole-3-carbinol | 77.375 |
| thapsigargin | 80 |
| diphenyleneiodonium | 83.5 |
| parthenolide | 89 |
| artesunate | 91.5 |
| chlorpromazine | 93.625 |
| pifithrin | 98 |
| ivermectin | 102.25 |
| cycloheximide | 102.375 |
| fluoxetine | 102.5 |
| clozapine | 103.375 |
| ethambutol | 104.75 |
| selamectin | 110.625 |
| triclosan | 115.375 |
| tunicamycin | 117 |
| mifepristone | 119.875 |
| genistein | 126.625 |
| cinobufagin | 126.875 |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

1-51. (canceled)

52. A method of treating a symptom of a neurodevelopmental disorder in a subject in need thereof, the method comprising administering to the subject a therapeutically effective amount of an agent of Table 1.

53. The method of claim 52, comprising administering to the subject a therapeutically effective amount of vorinostat or ivermectin.

54. The method of claim 52, wherein the neurodevelopmental disorder is Rett syndrome.

55. The method of claim 52, wherein the neurodevelopmental disorder is CDKL5 deficiency disorder.

56. The method of claim 52, where the subject has Rett syndrome or has been diagnosed with Rett syndrome and is treated with a therapeutically effective amount of vorinostat.

57. The method of claim 56, wherein vorinostat is formulated with hydroxypropyl beta-cyclodextrin.

58. The method of claim 56, wherein the amount of vorinostat is effective to: reduce anxiety in the subject, relative to a control; improve Rett syndrome scoring of the subject, relative to a control; decrease inflammation in the subject, relative to a control; reduce, inhibit, or reverse ciliary dysfunction in the subject, relative to a control; or the amount of vorinostat is lower than the amount of vorinostat approved by the U.S. Food and Drug Administration (FDA) for the treatment of cutaneous T cell lymphoma (CTCL).

59. The method of claim 54, where the subject has Rett syndrome or has been diagnosed with Rett syndrome and is treated with a therapeutically effective amount of ivermectin.

60. The method of claim 56, wherein vorinostat is administered to the subject in combination with one or more of ivermectin, trofinetide, and Bacteroides fragilis or a polysaccharide isolated from Bacteroides fragilis.

61. The method of claim 58, wherein ivermectin is administered to the subject in combination with one or more of vorinostat, trofinetide, and Bacteroides fragilis or a polysaccharide isolated from Bacteroides fragilis.

62. The method of claim 55, wherein the subject has CDKL5 deficiency disorder or has been diagnosed with CDKL5 deficiency disorder.

63. The method of claim 62, the method comprising administering to the subject a therapeutically effective amount of vorinostat.

64. The method of claim 63, wherein vorinostat is formulated with hydroxypropyl beta-cyclodextrin.

65. The method of claim 63, wherein the amount of vorinostat is effective to: reduce anxiety in the subject, relative to a control; decrease inflammation in the subject, relative to a control; reduce, inhibit, or reverse ciliary dysfunction in the subject, relative to a control.

66. The method of claim 63, wherein the vorinostat is administered in combination with one or more of ivermectin, trofinetide, and Bacteroides fragilis or a polysaccharide isolated from Bacteroides fragilis.

67. The method of claim 62, the method comprising administering to the subject a therapeutically effective amount of ivermectin.

68. The method of claim 67, wherein ivermectin is administered in combination with one or more of vorinostat, trofinetide, and Bacteroides fragilis or a polysaccharide isolated from Bacteroides fragilis.

69. The method of claim 53, wherein vorinostat or ivermectin is administered weekly, daily, or multiple times a day.

70. A pharmaceutical composition comprising (a) two or more of vorinostat, ivermectin, trofinetide, and Bacteroides fragilis or a polysaccharide isolated from Bacteroides fragilis and (b) a pharmaceutically acceptable excipient.

71. The pharmaceutical composition of claim 70, wherein the excipient is hydroxypropyl beta-cyclodextrin.
